(19) United States
(12) Patent Application Publication
(10) Pub. No.: US 2014/0228765 A1
(43) Pub. Date: Aug. 14, 2014

(54) Needle Penetration Detection Method and Device for Refillable and Implantable Drug Delivery Systems

(71) Applicant: Flowonix Medical Incorporated, Mt. Olive, NJ (US)

(72) Inventors: Paul BURKE, Bellingham, MA (US); Steve ADLER, Randolph, NJ (US); Robert M. HANSEN, Fairfax, VA (US)

(73) Assignee: Flowonix Medical Incorporated, Mt. Olive, NJ (US)

(21) Appl. No.: 14/171,840

(22) Filed: Feb. 4, 2014

Related U.S. Application Data
(60) Provisional application No. 61/763,277, filed on Feb. 11, 2013.

(51) Int. Cl. A61M 5/142; G01N 27/02; A61M 39/04

(52) U.S. Cl. CPC A61M 5/14276; A61M 39/04; G01N 27/02
USPC 604/181; 324/656

(57) ABSTRACT

A refillable and implantable infusion apparatus and method that includes a needle penetration detector that detects and indications the position of a needle relative to a septum of a drug reservoir of the implantable infusion apparatus. With the needle position data, medical professionals may better ensure they are injecting drugs into the drug reservoir, thus, improving patient safety.

### RELATED APPLICATIONS

This application claims the benefit of priority to U.S. Provisional Application No. 61/763,277 entitled “Needle Penetration Detection Method and Device for Refillable and Implantable Drug Delivery Systems” filed Feb. 11, 2013, the entire contents of which are hereby incorporated by reference in their entirety for all purposes.

### FIELD

The present invention relates generally to implantable infusion devices for the delivery of medication or other fluids to a patient.

### BACKGROUND

Various implantable devices exist for delivering infusate, such as medication, to a patient. One such device is an implantable valve accumulator pump system. This system includes an electronically controlled metering assembly located between a drug reservoir and an outlet catheter. Doctors may refill the drug reservoir on a periodic basis (e.g., once a month) for the patient.

### SUMMARY

The systems, methods, and devices of the various embodiments provide an indication to a medical professional when an inserted needle has penetrated a septum of the drug reservoir of an implantable drug delivery device. The various embodiments may enable a medical professional to determine whether to inject drugs into the implantable drug delivery device based on an indication that the inserted needle is in the proper position. In an additional embodiment, the needle penetration detector and its associated devices may give assurances to medical professionals that drugs were delivered to the drug reservoir in the patient properly based on proper needle positioning

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIG. 1 is a schematic diagram of an implantable drug delivery system.

FIG. 2 is a schematic diagram of a needle penetration detector.

FIG. 3 is a graph of various changes in inductance versus the depth of an approaching needle as observed by a needle penetration detector.

FIG. 4 is a process flow diagram illustrating an embodiment method for detecting a needle in an implantable drug delivery system.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

The words “exemplary” or “for example” are used herein to mean “serving as an example, instance, or illustration.” Any implementation described herein as “exemplary” or “for example” is not necessarily to be construed as preferred or advantageous over other implementations.

The systems, methods, and devices of the various embodiments enable delivering metered doses of a drug or other infusate. An embodiment drug delivery system may include a needle penetration detector, an electronics module, and an external programmer to indicate to a medical professional refilling a drug reservoir of the drug delivery system a status of needle insertion into the drug reservoir. In an embodiment, the medical professional may activate the needle penetration detector with the external programmer. The electronics module connected to the needle penetration detector may measure the inductance in a coil surrounding the septum of the drug reservoir prior to insertion of a needle and after insertion of the needle into or near the septum. In an embodiment, a needle penetrating the septum may result in a change in the inductance of the coil compared to the inductance in the coil in normal conditions (i.e., when a needle is not present). In an embodiment, the electronics module may monitor the state of the inductance of the coil and the connected external programmer may indicate the proper or improper position based on the measured inductance of the coil.

Various embodiments may provide a needle penetration detector that indicates to a medical professional that he has successfully penetrated a needle into the drug reservoir to refill it. Alternatively, the needle penetration detector may indicate to the medical professional that he has not penetrated the needle into the drug reservoir and the medical professional may halt injecting the drug outside of the drug reservoir and re-attempt to penetrate the drug reservoir with the needle. In this way, patient safety is improved and the medical professional may have an instant verification that the needle is in proper position to inject drugs into the reservoir.

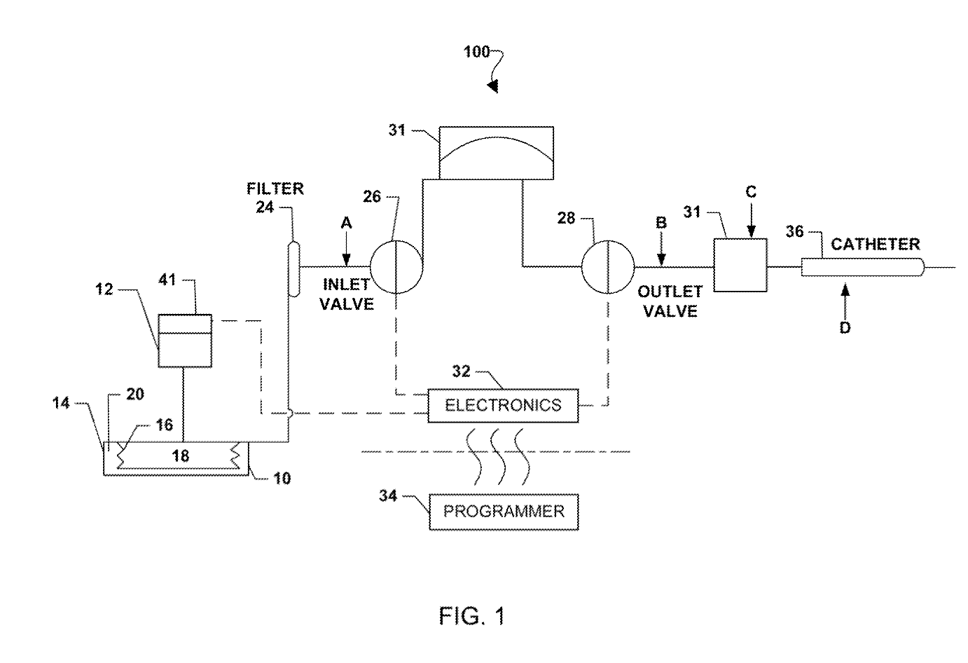

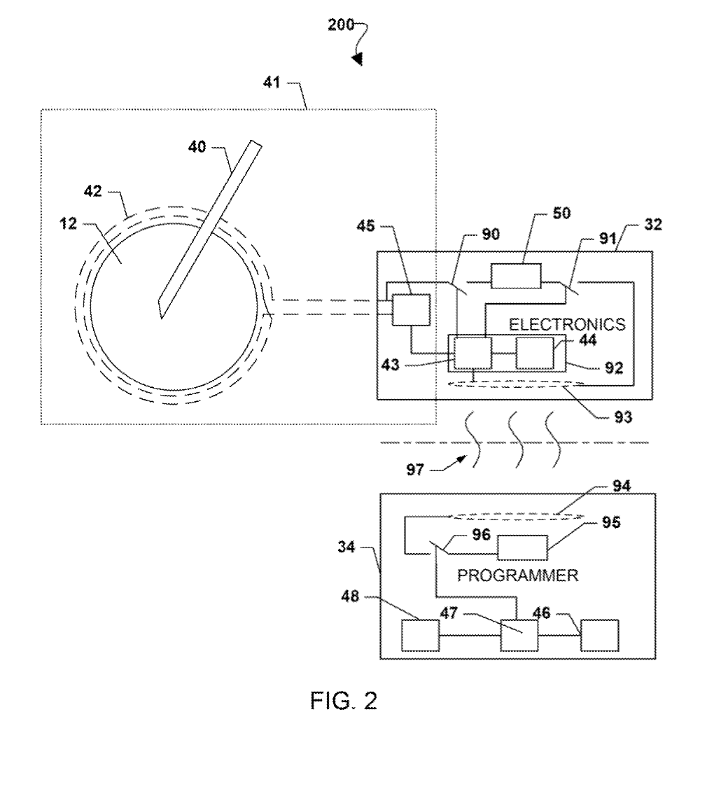

FIG. 1 illustrates an embodiment of an implantable valve accumulator pump system 100 for the delivery of infusate, such as medication. The system 100 may generally include four assemblies. The first major assembly is a rechargeable, constant pressure drug reservoir 10 in series with a bacteria/air filter 24. In one embodiment, the reservoir 10 comprises a sealed housing 14 containing a bellows 16. The bellows 16 separates the housing 14 into two parts. Chamber 18 is used to hold the drug or other medicinal fluid. Second zone 20 is normally filled with a two-phase fluid, such as Freon®, that has a significant vapor pressure at body temperature. Thus, as the fluid within the second zone 20 vaporizes, it compresses the bellows 16, thereby pressurizing the drug in the chamber 18. The drug can be refilled via a refill septum 12. The induction coil 42 (shown in FIG. 2) of the needle penetration detector 41 may surround the refill septum 12 and may be electrically controlled by a processor 43 the electronics module 32. The electronic module 32 may be programmed via an external programmer 34.

The two-phase fluid helps maintain the chamber 18 under a constant pressure. When the chamber is refilled, the two-phase fluid is pressurized thereby condensing a portion of the vapor and converting it to liquid. As the chamber 18 is emptied, this liquid vaporizes, thus maintaining the pressure on the bellows 16. Since the infusate in chamber 18 is under positive pressure, it is urged out of the chamber, through a bacterial filter 24 and toward the metering assembly.

The second major assembly is an electronically controlled metering assembly comprising two normally closed solenoid valves 26, 28, which are positioned on the inlet and outlet sides of a fixed volume accumulator 30. The valves are controlled electronically via an electronics module 32, which may be programmed utilizing the external programmer 34. The metering assembly is designed such that the inlet valve 26 and the outlet valve 28 are never simultaneously open.

The third major assembly is an outlet catheter 36 for medication infusion in a localized area. The delivery of fluid occurs at an infusion site that is below the accumulator pressure, thereby forcing discharge through the catheter 36.

The drug reservoir, electronically controlled metering assembly, and needle penetration detector may be contained within a biocompatible housing, also containing a power source (e.g., battery), that may be implanted within the body of a human or animal patient. The outlet catheter may be integral with the housing, or may be a separate component that is attached to the housing. An access port 31, in communication with the catheter 36, may be provided downstream of the metering assembly. The access port 31 may be used, for example, to manually provide a bolus dose of medication to the patient.

The fourth assembly of the system of FIG. 1 is the external programmer 34 used to communicate and program the desired medication regimen and to activate and/or control the needle penetration detector 41. In an embodiment, the external programmer 34 may be a handheld unit with a touch screen. The external programmer 34 may provide a wireless data transfer link to a wireless communication transceiver within the implanted electronics module 32 and may be enabled to exchange information with the electronic module 32, including but not limited to battery status, diagnostic information, calibration information, etc. In an embodiment, the external programmer 34 may send an activation instruction to the electronics module 32 to activate the electronics module 32. In an embodiment, the external programmer 34 may indicate to a medical professional the position of an inserted needle by receiving an indication instruction from the electronics module 32 which may indicate the needle's location relative to the refill septum 12. In an embodiment, the electronics module 32 may include a coil configured to send and receive electromagnetic signals to/from the external programmer 34.

FIG. 2 illustrates an implantable drug delivery device 200 that includes an embodiment needle penetration detector 41. In an embodiment, the refill septum 12 may be surrounded by an induction coil 42 of the needle penetration detector 41. A medical professional may pierce a needle 40 through the refill septum 12 surrounded by the induction coil 42. The electronics module 32 may include a controller 92. In an embodiment, the controller 92 may include a processer 43 coupled to a memory 44. The processor 43 may be any type of programmable processor, such as a microprocessor or microcontroller, which may be configured with processor-executable instructions to perform the operations of the embodiments described herein. Processor-executable software instructions may be stored in the memory 44 before they are accessed and loaded into the processor 43. The processor 43 may include internal memory sufficient to store the application software. The memory 44 may be volatile, nonvolatile such as flash memory, or a mixture of both. The electronics module 32 may include an alternating current (AC) power source 50. The AC power source 50 may be coupled to a wireless communication coil 93 of the electronics module 32 via a switch 91 coupled to the controller 92. The controller 92, particularly the processor 43, may control the operation of the switch 91 to induce a modulated magnetic field on coil 93 to communicate information to and receive commands and configuration data from a programmer 34 via a wireless communication link 97. The use of modulated magnetic fields to induce currents in induction coils to communicate with implanted medical devices is well known. For example, currents flowing through the wireless communication coil 93 may be modulated by the controller or a dedicated wireless communication transceiver to induce currents in a wireless communication coil 94 in an external programmer 34 to communicate information to the external programmer 34, and vice versa. The controller 92 may be coupled to the wireless communication coil 93 and may monitor the current, voltage, and/or inductance of the coil 93 and function as a wireless communication transceiver to receive information via the wireless communication coil 93 from an external programmer 34. As an example, the controller 92 may receive operational configuration information such as a dosage regimen via the wireless communication coil 93 from the external programmer 34.

In an embodiment, the controller 92 may be coupled to an inductance monitoring circuit 45 of the needle penetration detector 41. The inductance monitoring circuit 45 may measure the inductance of the induction coil 42 and provide indications of the measurements of the inductance to controller 92. The AC power source 50 may be coupled to the induction coil 42 via a switch 90 coupled to the controller 92. The controller 92 may control the operation of the switch 90 to induce a magnetic field on coil 12.

In an embodiment, the inductance monitoring circuit 45 may measure the inductance of the induction coil 42 resulting from the change in inductance from an approaching needle and provide the measurement of the inductance to the controller 92. The controller 92 may compare the change in inductance from the approaching needle to an established baseline inductance. The controller 92 may determine the needle's position and communicate that determination to the external programmer 34, which may subsequently indicate the position of the needle 40. In another embodiment, the controller 92 may generate indications of the measurements of the inductance received from the inductance monitoring circuit 45 and communicate the indications of the measurements to the external programmer 34.

In an embodiment, the external programmer 34 may include a processor 47 coupled to a memory 46 and to an indicator 48. Software instructions may be stored in the memory 46 before they are accessed and loaded into the processor 47. The external programmer 34 may include an AC power source 95 coupled to a wireless communication coil 94 via a switch 96 coupled to the processor 47. The processor 47 may control the operation of switch 96 to induce a magnetic field on the wireless communication coil 94 to receive and communicate information. For example, the wireless communication coil 94 may be controlled to communicate information from the electronics module 32. The processor 47 may be coupled to the wireless communication coil 94 and may monitor the current, voltage, and/or inductance of the coil 94 to receive information from the via the wireless communication coil 94. As an example, the processor 47 may receive information via the wireless communication coil 94 from the electronics module 32 of the implantable drug delivery device 200 regarding whether a needle has been detected within the induction coil 42. The processor 47 may be connected to an indicator 48 to indicate the position of a needle based on received indication instructions from the electronics module 32. For example, the indicator may be a display, a speaker for an audio sound or message, or a vibrator to generate haptic feedback.

In an embodiment, the external programmer 34 may receive, via the wireless communication link 97 described above, information regarding the position of the needle 40 and/or indications of the measurements of the inductance of the induction coil 42 from the implantable drug delivery device 200. The information communicated from the implantable drug delivery device 200 may be a direct measure of inductance of the induction coil 42 or data that the external programmer processor 47 can used to determine changes in inductance. For example, the processor 47 may compare the change in inductance from the approaching needle 40 to a baseline inductance established before the needle 40 was inserted into the patient to detect when the needle 40 is in a proper or improper position. Alternatively, the processor 43 of the implantable drug delivery device 200 may compare measurements of the inductance of the induction coil 42 and transmit to the external programmer 34 an indication of whether the needle 40 is in a proper or improper position. If the needle 40 is in an improper position, the external programmer 34 may inform the medical professional via the indicator 48, thereby allowing the medical professional to reposition the needle into the refill septum 12 of the drug reservoir.

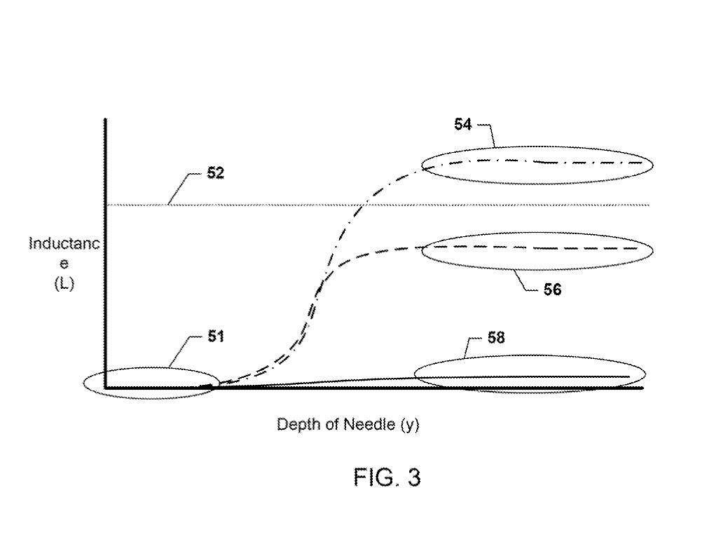

FIG. 3 illustrates a curve of the measured inductance (L) over the depth of a needle (y) observed by a needle penetration detector interacting with an approaching needle. In an embodiment, the inductance monitor 45 may measure the inductance of coil 42 of a needle penetration detector prior to inserting a needle into a patient to establish a baseline inductance measurement. The graph of inductance (L) over the depth of a needle (y) illustrated in FIG. 3 illustrates the relative change in inductance (L) from the baseline inductance established before the insertion of as needle into a patient and the change in inductance (L) which may indicate the position of a needle. When AC power is applied to the induction coil of the needle penetration detector the inductance (L) but the needle is not inserted in the induction coil, the inductance in the induction coil may be measured as a baseline inductance indicated by inductance measurement region 51. When a needle is inserted into the patient a first far distance away from the induction coil of the needle penetration detector and the AC power is applied to the induction coil of the needle penetration detector, the inductance in the induction coil may exceed the baseline inductance by a relatively small value as indicated by inductance measurement region 58. When a needle is inserted to a second distance closer to the induction coil of the needle penetration detector (e.g., inserted deeper into the patient toward the coil) and the AC power is applied to the induction coil of the needle penetration detector the inductance in the induction coil may exceed the baseline inductance by a larger value as indicated by inductance measurement region 56. When the needle is inserted in the refill septum (i.e., into the induction coil of the needle penetration detector surrounding the refill septum) and the AC power is applied to the induction coil of the needle penetration detector the inductance in the induction coil may be a high inductance value as indicated by inductance measurement region 58. In an embodiment, a high inductance value exceeding a threshold inductance 52 may indicate that the needle is positioned in the septum.

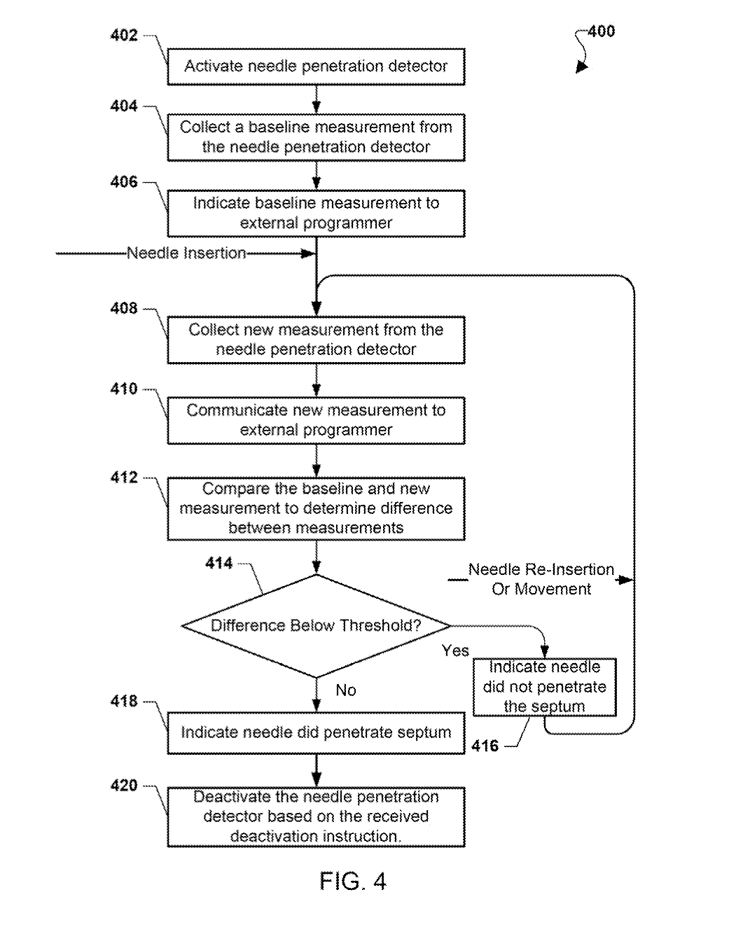

FIG. 4 illustrates an embodiment method 400 for detecting a position of a needle in a refill septum of an implantable drug delivery device. The electronics module 32 determines the position of a penetrating needle relative to the refill septum 12 of the drug reservoir. In block 402 an electronics module 32 of the implantable drug delivery device may activate the needle penetration detector 41. In an embodiment, an electronics module 32 may receive an activation instruction to activate a needle penetration detector 41 from an external programmer 34. For example, a medical professional may use the external programmer 34 to send the activation instructions to the electronics module 32 within the implantable drug delivery device implanted within the patient. In an embodiment, the processor 43 of the electronics module 32 within the implantable drug delivery device may periodically activate the needle penetration detector by sending electricity to the induction coil 42. For example, the electronics module 32 may turn on the needle penetration detector every second, quarter second, one-hundredth of a second, microsecond, five microseconds, millisecond, etc.

In block 404 the induction monitor 45 of the electronics module may collect a baseline measurement from the needle penetration detector 41. For example, the electronics module 32 may collect a baseline measurement of the inductance of the induction coil 42 before a needle is inserted into the refill septum. In block 406 the electronics module 32 may indicate the baseline measurement to the external programmer 34. For example, the electronics module 32 may transmit the baseline measurement to the external programmer 34 via the coils 93, 94.

In block 408 the induction monitor 45 of the electronics module 32 may collect a new measurement from the needle penetration detector 41. The new measurement may be collected after the medical professional inserts a needle into the patient. In block 410, the electronics module 32 may communicate the new inductance measurement to the external programmer 34. In block 412, a processor of the external programmer 34 may compare the baseline and new measurements to determine the difference between measurements. The comparison may result in a high state 54, an intermediate state 56, or a low state 58 as shown in FIG. 3. For example, the processor 47 of the external programmer 34 may compare the difference in inductance between the baseline measurement and the new measurement to one or more threshold values, such as threshold values associated with a high state 54 that indicates when the needle has penetrated the induction coil 42 of the refill septum.

In determination block 414, a processor 47 of the external programmer 34 may determine whether the difference in measurements is below a threshold 52. In response to determining that the difference in measurements is below the threshold 52 (i.e., determination block 414=“Yes”), the processor 47 of the external programmer 34 may indicate via an indicator 48 or display that the needle did not penetrate the refill septum in block 416. For example, a medical professional may have inserted the needle 40 outside of the refill septum 12 in which case the indicator 48 of the external programmer may display an appropriate warning or message, such as “FAIL.” Based on the indication, the medical professional may re-insert or move the needle and the electronics module and the external programmer may repeat blocks 408, 410, 412. In an embodiment, the processor 47 of the external programmer 34 may provide an intermediate indication via the indicator 48 when the measured inductance indicates that the needle is close but not yet within the refill septum. This intermediate indication may aid a medical professional in aligning the needle with the refill septum before penetrating the skin of the patient.

In response to determining that the difference in measurements is equal to or greater than the threshold 52 (i.e., determination block 414=“No”), the processor 47 of the external programmer 34 may indicate the needle did penetrate the septum in block 418. For example, a medical professional may have inserted the needle 40 directly in the center of the refill septum 12, in which case the indicator 48 of the external programmer may display an appropriate message, such as “SUCCESS.”

In alternative embodiment, the processor 43 within the implantable drug delivery device may be configured with processor-executable instructions to perform the operations of blocks 412 and 414 and communicate an indication of success or failure (and optionally an intermediate indication) to the external programmer 34. In this embodiment, the processor 47 of the external programmer 34 may receive the indication from the implantable drug delivery device and use the received indication to generate a corresponding warning or message on the indicator 48 or display.

In block 420, the electronics module 32 of the implantable drug delivery device may deactivate the needle penetration detector based on a received deactivation instruction from the external programmer 34. For example, a medical professional may press a button labeled “Deactivate” on the external programmer 34, which prompts the external programmer to send a deactivate instruction via coil 94 to coil 93 of the electronics module 32 of the implantable drug delivery device. Upon receiving the deactivation instructions, the electronics module of the implantable drug delivery device may cut off electricity to the induction coil 42 of the needle penetration detector 41, thereby conserving the battery life of the electronics module 32.

The foregoing method descriptions and the process flow diagrams are provided merely as illustrative examples and are not intended to require or imply that the blocks of the various aspects must be performed in the order presented. As will be appreciated by one of skill in the art the order of blocks in the foregoing aspects may be performed in any order. Words such as “thereafter,” “then,” “next,” etc. are not intended to limit the order of the blocks; these words are simply used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm blocks described in connection with the aspects disclosed herein may be implemented as electronic hardware, computer software, or combinations of both. To clearly illustrate this interchangeability of hardware and software, various illustrative components, blocks, modules, circuits, and blocks have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A method for detecting a needle penetration into a septum of a refillable implantable drug delivery system, comprising:
obtaining a baseline measurement of inductance of a needle penetration detector within the septum of a refillable implantable drug delivery system prior to a needle penetration of a patient;
obtaining a measurement of the inductance of the needle penetration detector following a needle penetration of the patient;
comparing the measurement of inductance to the baseline measurement of inductance;
determining whether the difference between the baseline measurement of inductance and the measurement of inductance satisfies a threshold;
indicating the needle did not penetrate the septum in response to determining that the difference between the baseline measurement and the measurement of inductance not satisfy the threshold; and
indicating the needle did penetrate the septum in response to determining that the difference between the baseline measurement and the measurement of inductance satisfies the threshold.

2. A refillable implantable drug delivery system, comprising:
a refill septum;
an induction coil surrounding the refill septum;
an inductance measuring circuit connected to the induction coil;
a wireless communication transceiver; and
a processor coupled to the inductance measuring circuit and the wireless communication transceiver, wherein the processor is configured with processor-executable instructions to perform operations comprising:
obtaining a base line measurement of inductance of the induction coil;
obtaining a measurement of the inductance of the induction coil;
determining whether a difference between the baseline measurement of inductance and the measurement of inductance satisfies a first threshold;
transmitting a signal via the wireless communication transceiver indicating that a needle did not penetrate the refill septum in response to determining that the difference between the baseline measurement and the measurement of inductance does not satisfy the first threshold; and
transmitting a signal via the wireless communication transceiver indicating that the needle did penetrate the refill septum in response to determining that the difference between the baseline measurement and the measurement of inductance satisfies the first threshold.

3. The refillable implantable drug delivery system of claim 2, wherein the processor is configured with processor-executable instructions to perform operations further comprising transmitting a signal via the wireless communication transceiver indicating that the needle is near the septum upon determining that the difference between the baseline measurement and the measurement of inductance satisfies a second threshold that is different from the first threshold.

4. A refillable implantable drug delivery system, comprising:
a refill septum;
an induction coil surrounding the refill septum;
means for obtaining a base line measurement of inductance of the induction coil;
means for obtaining a measurement of the inductance of the induction coil;
means for determining whether a difference between the baseline measurement of inductance and the measurement of inductance satisfies a first threshold;
means for transmitting a signal indicating that a needle did not penetrate the refill septum in response to determining that the difference between the baseline measurement and the measurement of inductance does not satisfy the first threshold; and
means for transmitting a signal indicating that the needle did penetrate the refill septum in response to determining that the difference between the baseline measurement and the measurement of inductance satisfies the first threshold.

5. The refillable implantable drug delivery system of claim 4, further comprising:
means for transmitting a signal indicating that the needle is near the refill septum upon determining that the difference between the baseline measurement and the measurement of inductance satisfies a second threshold that is different from the first threshold.
